(19) United States
(12) Patent Application Publication
(10) Pub. No.: US 2004/0014740 A1
(43) Pub. Date: Jan. 22, 2004

(54) Novel anthelmintic and insecticidal compositions

(76) Inventors: Byung Hyun Lee, Kalamazoo, MI (US); Timothy G. Geary, Kalamazoo, MI (US); John P. Davis, Kalamazoo, MI (US); Jerry W. Bowman, Lawton, MI (US)

Correspondence Address: PHARMACIA & UPJOHN, 301 HENRIETTA ST, 0228-32-LAW, KALAMAZOO, MI, 49007, US

(21) Appl. No.: 10/446,253

(22) Filed: May 28, 2003

Related U.S. Application Data
(60) Provisional application No. 60/385,017, filed on May 31, 2002.

(51) Int. Cl. A61K 31/453; A61K 31/397; C 07D 4 9/02

(52) U.S. Cl. 514/210.19; 514/326; 514/342; 546/212; 546/280.4; 548/950

(57) ABSTRACT

Novel anthelmintic compositions containing thiophene derivatives as active ingredients are disclosed.

### CROSS REFERENCE TO RELATED APPLICATIONS

This application claims the benefit of U.S. provisional application Serial No. 60/385,017, filed May 31, 2002, under 35 USC 119(e)(i), which is incorporated herein by reference in its entirety.

### BACKGROUND OF THE INVENTION

1. Field of the Invention

The present invention relates to novel anthelmintic and insecticidal compositions in general, and, more specifically, compositions containing pyrazole derivatives as active ingredients.

2. Technology Description

Control of parasitic infections in human and animal populations remains an important global endeavor. The causative organisms may be categorized as endoparasitic members of the classes Nematoda, Cestoidea and Trematoda or phylum Protozoa, or as ectoparasitic members of the phylum Arthropoda. The former comprises infections of the stomach, intestinal tracts, lymphatic system, tissues, liver, lungs, heart and brain. Examples include trichinosis, lymphatic filariasis, onchocerciasis, schistosomiasis, leishmaniasis, trypanosomiasis, giardiasis, coccidiosis and malaria. The latter ectoparasites include lice, ticks, mites, biting flies, fleas and mosquitoes. These often serve as vectors and intermediate hosts to endoparasites for transmission to human or animal hosts. While certain helminthiases can be treated with known drugs, evolutionary development of resistance necessitates a further search for improved efficacy in next generation anthelmintic agents.

The control of ectoparasites, such as fleas, ticks, biting flies and the like, has long been recognized as an important aspect of human and animal health regimens. Traditional treatments were topically applied, such as the famous dips for cattle, and indeed such treatments are still in wide use. The more modern thrust of research, however, has been towards compounds, which can be administered orally, or parenterally to the animals and which will control ectoparasitic populations by poisoning individual parasites when they ingest the blood of a treated animal.

The control of endoparasites, especially intestinal parasites, has also been an important aspect of human and animal health regimens. Although a number of ectoparasiticides and endoparasiticides are in use, these suffer from a variety of problems, including a limited spectrum of activity, the need for repeated treatment and, in many instances, resistance by parasites. The development of novel endo- and ectoparasiticides is therefore essential to ensure safe and effective treatment of a wide range of parasites over a long period of time.

Despite the above teachings, there still exists a need in the art for treatment of pests.

### BRIEF SUMMARY OF THE INVENTION

In accordance with the present invention, a novel composition of matter that is capable of treatment of pests is provided. The composition contains thiophene derivatives as active ingredients.

A first embodiment of the present invention provides a compound of Formula I comprising:

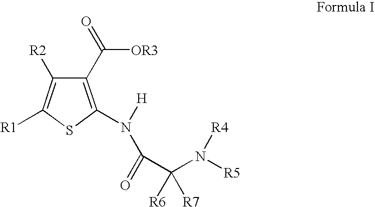

wherein R₁ and R₂ are selected from the group consisting of H, alkyl, phenyl, substituted phenyl, benzyl, substituted benzyl, heteroaryl, substituted heteroaryl, hetroarylmethylene, and substituted hetroarylmethylene; or

R₁ and R₂, along with the carbons to which they are attached, form a 5 to 7 membered substituted or unsubstituted carbocyclic or heterocyclic ring;

R₃ is alkyl of 1 to 4 carbons;

R₄, and R₅ are independently alkyl, heteroalkyl, cycloalkyl, aryl, aralkyl, heteroaryl or heteroaralkyl;

R₄ and R₅ taken together may form a 4-7 membered substituted or unsubstituted carbocyclic ring;

R₆ and R₇ are independently H or alkyl of 1 to 3 carbons.

### DETAILED DESCRIPTION OF THE INVENTION

Definitions

The term “alkyl,” by itself or as part of another substituent, means, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e. C₁-C₈ means 1-8 eight carbons). Examples of saturated hydrocarbon radicals include groups such as methyl, ethyl, n-propyl, isopropyl, n-butyl, t-butyl, isobutyl, sec-butyl, cyclohexyl, (cyclohexyl)ethyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkylene” by itself or as part of another substituent means a divalent radical derived from an alkane, as exemplified by —CH₂CH₂CH₂CH₂—. A “lower alkyl” or “lower alkylene” is a shorter chain alkyl or alkylene group, having eight or fewer carbon atoms.

The terms “alkoxy . . . alkylcylamino” and “alkylthio” refer to those groups having an alkyl group attached to the remainder of the molecule through an oxygen, nitrogen or sulfur atom, respectively. Similarly, the term “dialkylamino” is used in a conventional sense to refer to —NR′R″ wherein the R groups can be the same or different alkyl groups.

The term “heteroalkyl,” by itself or in combination with another term, means, unless otherwise stated, a stable straight or branched chain, or cyclic hydrocarbon radical, or combinations thereof, fully saturated or containing from one to three degrees of unsaturation, consisting of the stated number of carbon atoms and from one to three heteroatoms selected from the group consisting of O, N, and S, and wherein the nitrogen and sulfur atoms may optionally be oxidized and the nitrogen heteroatom may optionally be quaternized. The heteroatom(s) O, N and S may be placed at any interior position of the heteroalkyl group. Examples include —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃. Also included in the term “heteroalkyl” are those radicals described in more detail below as “heterocycloalkyl.” The term “heteroalkylene” by itself or as part of another substituent means a divalent radical derived from heteroalkyl, as exemplified by —CH₂—CH₂—S—CH₂CH₂— and —CH₂—S—CH₂CH₂—NH—CH₂. For heteroalkylene groups, heteroatoms can also occupy either or both of the chain termini. Still further, for alkylene and heteroalkylene linking groups, no orientation of the linking group is implied.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl. include cyclopentyl, cyclohexyl, 1-cyclohexenyl, 3-cyclohexenyl, cycloheptyl, and the like. Examples of heterocycloalkyl include 1-piperidinyl, 2-piperidinyl, 3-piperidinyl, 4-morpholinyl, 3morpholinyl, tetraliydrofuran-2-yl, tetrahydrofuran-3-yl, tetrahydrothien-2-yl, tetrahydrothien-3-yl, 1-piperazinyl, 2-piperazinyl, and the like.

The terms “halo” or “halogen,” by themselves or as part of another substituent, mean, unless otherwise stated, a fluorine, chlorine, bromine, or iodine atom. Additionally, terms such as “Fluoroalkyl,” are meant to include monofluoroalkyl and polyfluoroalkyl.

The term “aryl,” employed alone or in combination with other terms (e.g., aryloxy, arylthioxy, aralkyl) means, unless otherwise stated, an aromatic substituent which can be a single ring or multiple rings (up to three rings) which are fused together or linked covalently. The term “heteroaryl” is meant to include those aryl rings which contain from zero to four heteroatoms selected from N, O, and S, wherein the nitrogen and sulfur atoms are optionally oxidized, and the nitrogen atom(s) are optionally quaternized. The “heteroaryl” groups can be attached to the remainder of the molecule through a heteroatom. Non-limiting examples of aryl and heteroaryl groups include phenyl, 1-naphthyl, 2-naplithyl, 4-biphenyl, 1-pyrrolyl, 2-pyrrolyl, 3-pyrroyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5thiazolyl, 2-furyl, 3-furyl, 2-thienyl, 3- thienyl, 2,7pyridyl, 3-pyridyl, 4-pyridyl, 2-pyrimidyl, 4-pyrimidyl, 5-benzothiazolyl, purinyl, 2-benzimidazolyl, 5-indolyl, lisoquinolyl, 5-isoquinolyl, 2-quinoxalinyl, 5-quinoxalinyl, 3-quinolyl, and 6-quinolyl.

Substituents for each of the above noted aryl ring systems are selected from the group of acceptable substituents described below. The term “aralkyl” is meant to include those radicals in which an aryl or heteroaryl group is attached to an alkyl group (e.g., benzyl, phenethyl, pyridylmethyl and the like) or a heteroalkyl group (e.g., phenoxymethyl, 2-pyridyloxymethyl, 3-(1-naphthyloxy)propyl, and the like). Each of the above terms (e.g., “alkyl . . . heteroalkyl” and “aryl”) are meant to include both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be a variety of groups selected from: —OR′, ═O, ═NR′, ═N—OR′, —NR′R″—SR′, -halogen, —SiR′R″R, —OC(O)R′, —C(O)R′, —CO2R′, CONR′R″, —OC(O)NR′R″—NR′C(O)R′, —NR′—C(O)NR″R′″, —NR′COOR′, —NH—C(NH₂)═NH, —NR′C(NH₂)═N—H, —NH—C(NH₂)—NR′, —S(O)R′, S(O)₂R′, —S(O)₂NR′R″, —CN and —NO₂ in a number ranging from zero to (2N+1), where N is the total number of carbon atoms in such radical. R′, R″ and X″ each independently refer to hydrogen, unsubstituted (Cl—COalkyl and heteroalkyl, unsubstituted aryl, aryl substituted with 1-3 halogens, unsubstituted alkyl, alkoxy or thioalkoxy groups, or aryl-(C₁-C₄)alkyl groups. When R′ and R″ are attached to the same nitrogen atom, they can be combined with the nitrogen atom to form a 5-, 6-, 7 or 7-membered ring. For example, —NR′R″ is meant to include 1-pyrrolidinyl and 4morpholinyl. From the above discussion of substituents, one of skill in the art will understand that the term “alkyl” is meant to include groups such as haloalkyl (e.g., —CF₃ and —CH₂CF₃) and acyl (e.g., —C(O)CH₃, —C(O)CF₃, —C(O)CH₂OCH₃, and the like).

Similarly, substituents for the aryl groups are varied and are selected from: halogen, —OR′, —OC(O)R′, —NR′R″, —SR′, —R′, —CN, —NO₂, —CO₂R′, —CONR′R:′, —C(O)R′, —OC(O)NR′R:′, —NR″C(O)R′, —NR″C(O)₂R′, —NR′—C(O)NR″R′″, —NH—C(NH₂)═NH, —NR′C(NH₂)═NH, —NH—C(NH₂)═NR′, —S(O)R′, —S(O)₂R′, —S(O)₂NR′R″—N₃, —CH(Ph)₂, perfluoro(C₁-C₄)alkoxy, and perfluoro(C₁-C₄)alkyl, in a number ranging from zero to the total number of open valences on the aromatic ring system; and where R′, R″ and R′″ are independently selected from hydrogen, (C₁-C₈)alkyl and heteroalkyl, unsubstituted aryl, (unsubstituted aryl)-(C₁-C₄)alkyl, and (unsubstituted arylOXY-(C₁-C₄)alkyl.

Two of the substituents on adjacent atoms of the aryl ring may optionally be replaced with a substituent of the formula -T-C(O)—(CH₂)q-U—, wherein T and U are independently —NH—, —O—, —CH₂— or a single bond, and the subscript q is an integer of from zero to two. Alternatively, two of the substituents on adjacent atoms of the aryl ring may optionally be replaced with a substituent of the formula -A-(CH₂),—B—, wherein A and B are independently —CH₂—, —O—, —NH—, —S—, —S(O)—, —S(O)₂—, —S(O)₂NR′— or a single bond, and r is an integer of from one to three. One of the single bonds of the new ring so formed may optionally be replaced with a double bond. Alternatively, two of the substituents on adjacent atoms of the aryl ring may optionally be replaced with a substituent of the formula —(CH₂),—X—(CH₂)t-, where s and t are independently integers of from zero to three, and X is —O—, —NR′—, —S—, —S(O)—, —S(O)₂—, or —S(O)₂NR′—. The substituent R′ in —NR′— and —S(O)₂NR′ is selected from hydrogen or unsubstituted (C₁-C₆)alkyl.

As used herein, the term “heteroatom” is meant to include oxygen (O), nitrogen (N), and sulfur(S).

The term “pharmaceutically acceptable salts” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactouronic acids and the like (see, for example, Berge et al. (1977) J. Miami. Sci.66:1-19). Certain specific compounds of the present invention contain both basic and acidic functionalities that allow the compounds to be converted into either base or acid addition salts.

The neutral forms of the compounds may be regenerated by contacting the salt with a base or acid and isolating the parent compound in the conventional manner.

The parent form of the compound differs from the various salt forms in certain physical properties, such as solubility in polar solvents, but otherwise the salts are equivalent to the parent form of the compound for the purposes of the present invention.

In addition to salt forms, the present invention provides compounds that are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex-vivo environment. For example, prodrugs can be slowly converted to the compounds of the present invention when placed in a transdermal patch reservoir with a suitable enzyme or chemical reagent. Prodrugs are often useful because, in some situations, they may be easier to administer than the parent drug. They may, for instance, be bioavailable by oral administration whereas the parent drug is not. The prodrug may also have improved solubility in pharmacological compositions over the parent drug. A wide variety of prodrug derivatives are known in the art, such as those that rely on hydrolytic cleavage or oxidative activation of the prodrug. An example, without limitation, of a prodrug would be a compound of the present invention that is administered as an ester (the “prodrug”), but then is metabolically hydrolyzed to the carboxylic acid, the active entity. Additional examples include peptidyl derivatives of a compound of the invention.

Certain compounds of the present invention can exist in unsolvated forms as well as solvated forms, including hydrated forms. In general, the solvated forms are equivalent to unsolvated forms and are intended to be encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, geometric isomers and individual isomers are all intended to be encompassed within the scope of the present invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

A first embodiment of the present invention provides a compound of Formula I comprising:

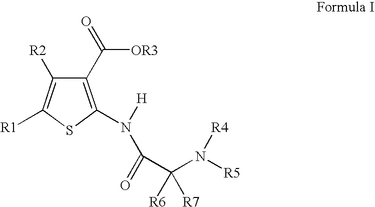

wherein R₁ and R₂ are selected from the group consisting of H, alkyl, phenyl, substituted phenyl, benzyl, substituted benzyl, heteroaryl, substituted heteroaryl, hetroarylmethylene, and substituted hetroarylmethylene; or

R₁ and R₂, along with the carbons to which they are attached, form a 5 to 7 membered substituted or unsubstituted carbocyclic or heterocyclic ring;

R₃ is alkyl of 1 to 4 carbons;

R₄, and R₅ are independently alkyl, heteroalkyl, cycloalkyl, aryl, aralkyl, heteroaryl or heteroaralkyl;

R₄ and R₅ taken together may form a 4-7 membered substituted or unsubstituted carbocyclic ring;

R₆ and R₇ are independently H or alkyl of 1 to 3 carbons.

A second embodiment of the present invention provides a composition comprising the compound of formula (I) and a carrier.

Another embodiment of the present invention comprises a process for the treatment or prevention of parasitic diseases in mammals, plants or agricultural crops comprising the step of administering to the mammal, plant or crop an effective amount of the above composition.

A further embodiment of the present invention comprises the use of the above-described composition to prepare a medicament for the treatment or prevention of parasitic diseases in mammals.

Yet another embodiment of the present invention comprises the above-described composition for use as a medicament.

An object of the present invention is to provide novel compositions that can be broadly used against parasites.

Still another object of the present invention is to provide a method for preventing or treating parasitic diseases in mammals by using a novel composition.

A further object of the present invention is to provide a method for producing a medicament using a novel composition.

These, and other objects, will readily be apparent to those skilled in the art as reference is made to the detailed description of the preferred embodiment.

The present invention is directed to the prevention and treatment of parasitic attack on host animals and provides a new tool for the control of parasitic organisms. In particular, the present invention provides a novel compound of formula (I):

In practice, the amount of the compound to be administered ranges from about 0.001 to 10 mg. per kg. of animal body weight, such total dose being given at one time or in divided doses over a relatively short period of time such as 1-5 days. Excellent control of such parasites is obtained in animals by administering from about 0.025 to 30 mg. per kg. of body weight in a single dose. Repeat treatments are given as required to combat re-infections and are dependent upon the species of parasite and the husbandry techniques being employed. The techniques for administering these materials to animals are known to those skilled in the veterinary field.

For use as an antiparasitic agent in animals the inventive composition may be administered internally either orally or by injection, or topically as a liquid drench or as a shampoo. These compositions may be administered orally in a unit dosage form such as a capsule, bolus or tablet. The drench is normally a solution, suspension or dispersion of the active ingredients usually in water together with a suspending agent such as bentonite and a wetting agent or like excipient. Generally, the drenches also contain an antifoaming agent. Drench formulations generally contains from about 0.01 to 10% by weight of each active compound. Preferred drench formulations may contain from 0.05 to 5.0% of each active by weight. The capsules and boluses comprise the active ingredients admixed with a carrier vehicle such as starch, talc, magnesium stearate, or di-calcium phosphate.

Where it is desired to administer the inventive composition in a dry, solid unit dosage form, capsules, boluses or tablets containing the desired amount of active compounds usually are employed. These dosage forms are prepared by intimately and uniformly mixing the active ingredient with suitable finely divided diluents, fillers, disintegrating agents and/or binders such as starch, lactose, talc, magnesium stearate, vegetable gums and the like. Such unit dosage formulations may be varied widely with respect to their total weight and content of the antiparasitic agent depending upon factors such as the type of host animal to be treated, the severity and type of infection and the weight of the host.

When the active composition is to be administered via an animal feedstuff it is intimately dispersed in the feed or used as a top dressing or in the form of pellets which may then be added to the finished feed or optionally fed separately. Alternatively, the antiparasitic compositions of the present invention may be administered to animals parenterally, for example, by intraruminal, intramuscular, intratracheal, or subcutaneous injection in which event the active ingredients are dissolved or dispersed in a liquid carrier vehicle. For parenteral administration, the active materials are suitably admixed with an acceptable vehicle, preferably of the vegetable oil variety such as peanut oil, cottonseed oil and the like. Other parenteral vehicles such as organic preparation using solketal, propylene glycol, glycerol formal, and aqueous parenteral formulations are also used, often in combination in various proportions. Still another carrier that can be selected is either N-methylpyrrolidone or 2-pyrrolidone and mixtures of the two. This formulation is described in greater detail in U.S. Pat. No. 5,773,442. To the extent necessary for completion, this patent is expressly incorporated by reference. The active compound or compounds are dissolved or suspended in the parenteral formulation for administration; such formulations generally contain from 0.005 to 5% by weight of each active compound.

In a particularly preferred embodiment, the carrier contains propylene glycol (1-99 percent by weight of the carrier) and glycerol formal (99-1 percent by weight of the carrier), with the relative amounts being 60% propylene glycol and 40% glycerol formal.

The present compositions may also be useful in yet another method in which the same active agents as above defined are employed as a “feed through larvicide.” In this method, the compound is administered to a vertebrate animal, especially a warm-blooded animal, in order to inhibit parasitic organisms which live in the feces of the animal. Such organisms are typically insect species in the egg or larval stage.

The inventive compositions are primarily useful as antiparasitic agents for the treatment and/or prevention of helminthiasis in all mammals, and preferably food animals and companion animals such as cattle, sheep, deer, horses, dogs, cats, goats, swine, and poultry. They are also useful in the prevention and treatment of parasitic infections of these animals by ectoparasites such as ticks, mites, lice, fleas and the like. They are also effective in the treatment of parasitic infections of humans. In treating such infections the inventive compositions may be used individually or in combination with each other or with other unrelated antiparasitic agents.

The exact dosage and frequency of administration of the inventive compositions depend on many factors, including (but not limited to) the severity of the particular condition being treated, the age, weight, and general physical condition of the particular patient (human or animal), and other medication the patient may be taking. These factors are well known to those skilled in the art, and the exact dosage and frequency of administration can be more accurately determined by measuring the concentration of the inventive composition in the patient's blood and/or the patient's response to the particular condition being treated.

The inventive compositions may also be used to combat agricultural pests that attack crops either in the field or in storage. The inventive compositions are applied for such uses as sprays, dusts, emulsions and the like either to the growing plants or the harvested crops. The techniques for applying the inventive compositions in this manner are known to those skilled in the agricultural arts.

Accordingly, it can be seen that the present methods can be utilized for protection against a wide range of parasitic organisms. Further, it should be noted that protection is achieved in animals with existing parasitic infections by eliminating the existing parasites, and/or in animals susceptible to attack by parasitic organisms by preventing parasitic attack. Thus, protection includes both treatment to eliminate existing infections and prevention against future infestations.

Representative parasitic organisms include the following:

Platyhelminthes:

Trematoda such as

Clonorchis

Echinostoma

Fasciola hepatica (liver fluke)

Fasciola gigantica

Fascioloides magna

Fasciolopsis

Metagonimus

Paragonimus

Schistosoma spp.

Nemathelminthes:

Ancylostoma

Angiostrongylus

Anisakis

Ascaris

Brugia

Bunostomum

Cooperia

Cyathostomum

Cylicocyclus

Dictyocaulus (lungworm)

Dipetalonema

Dirofilaria (heartworm)

Dracunculus

Elaeophora

Gaigeria

Globocephalus urosubulatus

Haemonchus

Metastrongylus (lungworm)

Muellerius (lungworm)

Necator americanus

Nematodirus

Oesophagostomum

Onchocerca

Ostertagia

Parascaris

Protostrongylus (lungworm)

Setaria

Stephanofilaria

Syngamus

Teladorsagia

Toxascaris

Toxocara

Trichinella

Trichostrongylus

Uncinaria stenocephala

Wuchereria bancrofti

Arthropoda:

Crustacea:

Argulus

Caligus

Arachnida:

Amblyomma americanum (Lone-star tick)

Amblyomma maculatum (Gulf Coast tick)

Argas persicus (fowl tick)

Boophilus microplus (cattle tick)

Demodex bovis (cattle follicle mite)

Demodex canis (dog follicle mite)

Dermacentor andersoni (Rocky Mountain spotted fever tick)

Dermacentor variabilis (American dog tick)

Dermanyssus gallinae (chicken mite)

Ixodes ricinus (common sheep tick)

Knemidokoptes gallinae (deplumming mite)

Knemidokoptes mutans (scaly-leg mite)

Otobius megnini (ear tick)

Psoroptes equi (scab mite)

Psoroptes ovis (scab mite)

Rhipicephalus sanguineus (brown dog tick)

Sarcoptes scabiei (mange mite)

Insecta:

Aedes (mosquito)

Anopheles (mosquito)

Culex (mosquito)

Culiseta (mosquito)

Bovicola bovis (cattle biting louse)

Callitroga hominivorax (blowfly)

Chrysops spp. (deer fly)

Cimex lectularius (bed bug)

Ctenocephalis canis (dog flea)

Ctenocephalis fells (cat flea)

Culicoides spp. (midges, sandflies, punkies, or no-see-ums)

Damalinia ovis (sheep biting louse)

Dermaobia spp. (warble fly)

Dermatophilus spp. (fleas)

Gasterophilus haemorrhoidalis (nose hot fly)

Gasterophilus intestinalis (common horse hot fly)

Gasterophilus nasalis (chin fly)

Glossina spp. (tsetse fly)

Haematobia irritans (horn fly, buffalo fly)

Haematopinus asini (horse sucking louse)

Haematopinus eurysternus (short nosed cattle louse)

Haematopinus ovilius (body louse)

Haematopinus suis (hog louse)

Hydrotaea irritans (head fly)

Hypoderma bovis (bomb fly)

Hypoderma lineatum (heel fly)

Linognathus ovillus (body louse)

Linognathus pedalis (foot louse)

Linognathus vituli (long nosed cattle louse)

Lucilia spp. (maggot fly)

Melophagus ovinus (sheep ked)

Oestrus ovis (nose hot fly)

Phormia regina (blowfly)

Psorophora

Reduviid bugs (assassin bug)

Simulium spp. (black fly)

Solenopotes capillatus (little blue cattle louse)

Stomoxys calcitrans (stable fly)

Tabanus spp. (horse fly)

Parasitic organisms that live in feces are typically the egg and larval stages of insects such as:

Musca domestica (housefly)

Musca autumnalis (face fly)

Haematobia spp. (horn fly, buffalo fly and others).

The invention is described in greater detail by the following non-limiting example.

### EXAMPLES

Compounds of the invention may be prepared by methods previously described (Perrissin, M. et.al., European Journal of Medicinal Chemistry, 1980, 15, 413; Gadad, A. K., et.al., Indian Journal of Chemistry, Section B, 1994, 33B, 298; A1-Obaid, et.al., Arzneimittel-Forschung, 1995, 45, 627) or according to the following general Schemes.

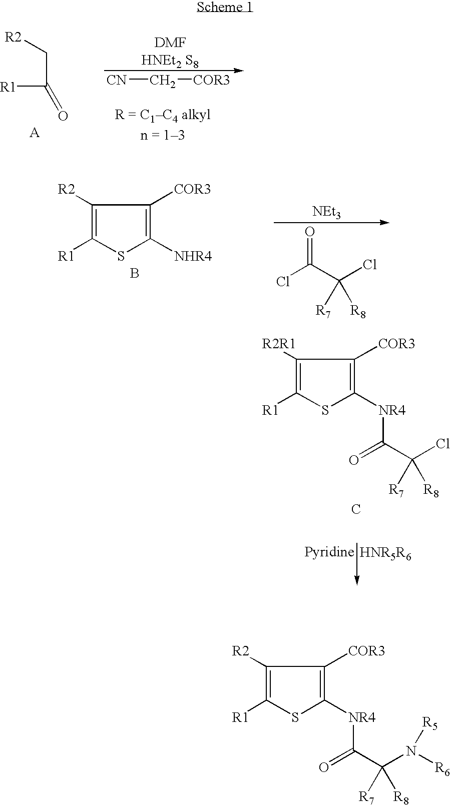

Ketone A is treated with a cyanoalkyl derivative and sulfur in the presence of triethyl amine and DMF to give B. Compound B is treated with acid chloride derivatives in the presence of triethyl amine to give C, which is treated with cyclic amines in the presence of pyridine to give the final products.

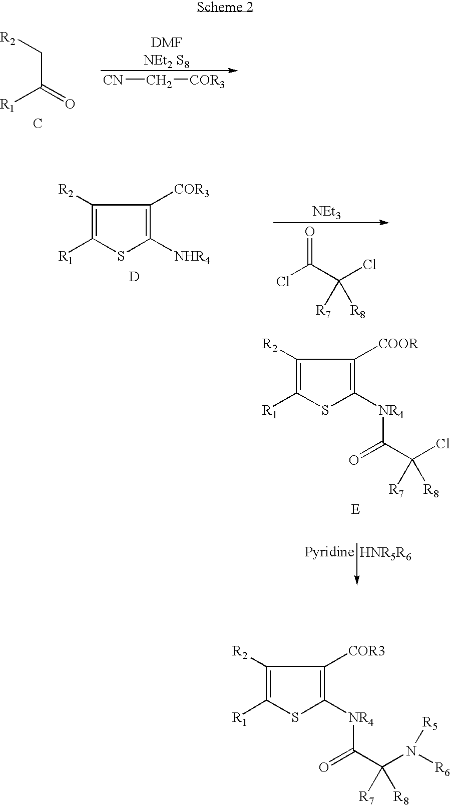

Ketone C is treated with cyanoalkyl derivative and sulfur in the presence of triethyl amine and DMF to give D. Compound D is treated with acid chloride derivatives in the presence of triethyl amine to give E, which is treated with cyclic amines in the presence of pyridine to give the final products.

### Specific Examples

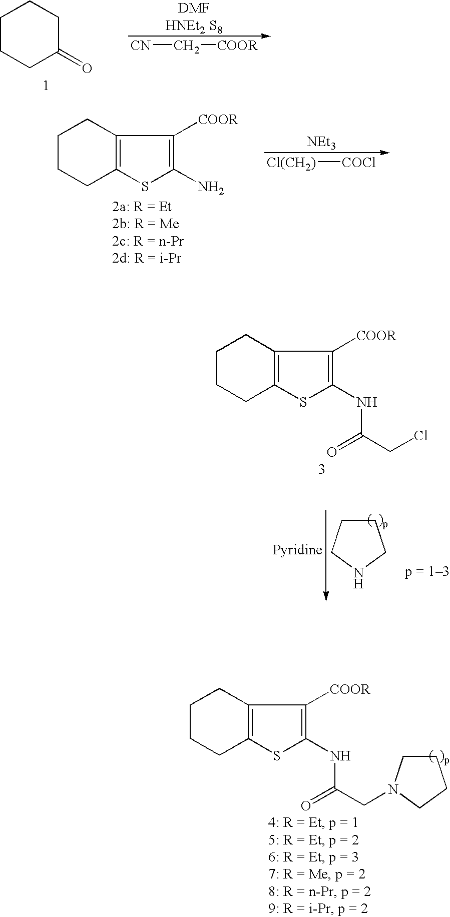

Preparation of 3a from 2a:

Compound 2a (1.13 g, 5 mmol) is dissolved in diethyl ether (30 mL) and treated with triethyl amine (1.8 mL, 12.5 mmol). To this mixture, chloroacetyl chloride (1.13 g, 10 mmol) dissolved in diethyl ether (5 mL) is added dropwise at room temperature. This mixture is stirred for 4 hours at room temperature. Insoluble material is filtered off and the filtrate is concentrated. The residue is triturated with ethanol (30 mL) and the precipitate is collected, washed with ethanol (2×10 mL) and dried to give a tan solid (0.93 g, 62% yield). Physical characteristics: MS (ES+) for m/z 301, 303 (M+H)⁺.

### Example 1

Preparation of 4:

Compound 3a (70 mg, 0.23 mmol) is dissolved in pyridine (2 mL) and treated with pyrrolidine (43 mg, 0.6 mmol). The mixture is heated to 80° C. for 2 hours. After pyridine is removed the residue is chromatographed on a silica plate by elution with 20% ethyl acetate in hexanes. The desired compound is isolated as a white solid (60 mg, 78% yield). Physical characteristics: MS (ES+) for m/z 337 (M+H)⁺; ¹H NMR (CDCl₃) δ 12.03, 4.33, 3.38, 2.78, 2.69, 2.64, 1.88, 1.78, 1.37.

### Example 2

Preparation of 5:

Compound 3a (100 mg, 0.33 mmol) is dissolved in pyridine (3 mL) and treated with piperidine (43 mg, 0.5 mmol). The mixture is heated to 80° C. for 2 hours. After pyridine is removed the residue is chromatographed on a silica plate by elution with 10% acetone in hexanes. The desired compound is isolated as a pale yellow solid (70 mg, 61% yield). Physical characteristics: MS (ES+) for m/z 351 (M+H)⁺; ¹H NMR (CDCl₃) δ 12.1, 4.36, 3.93, 3.422.6-2.9, 1.81, 1.64, 1.40.

### Example 3

Preparation of 6:

Compound 3a (70 mg, 0.23 mmol) is dissolved in pyridine (2 mL) and treated with hexamethyleneimine (43 mg, 0.44 mmol). The mixture is heated to 80° C. for 2 hours. After pyridine is removed the residue is chromatographed on a silica plate by elution with 20% ethyl acetate in hexanes. The desired compound is isolated as a white solid (60 mg, 78% yield). Physical characteristics: MS (ES+) for m/z 337 (M+H)⁺; ¹H NMR (CDCl₃) δ 12.23,4.34,3.35, 2.6-2.8, 1.78, 1.69, 1.37.

Preparation of 2b:

To methyl cyanoacetate (1.74 g, 17.6 mmol) and sulfur (0.56 g) in DMF (4 mL), diethylamine (1.4 mL) is added under stirring. To this solution cyclohexanone (1.73 g, 17.6 mmol) is added dropwise. The mixture is heated to 60° C. for 2 hours and it is poured into water (30 mL), which is extracted with diethyl ether (30 mL). The ethereal solution is dried with sodium sulfate and concentrated to give the desired compound as a yellow solid (0.6 g). Physical characteristics: MS (ES+) for m/z 210.

Preparation of 3b from 2b:

Compound 2b (0.50 g, 2.37 mmol) is dissolved in diethyl ether (10 mL) and treated with triethyl amine (1.8 mL, 12.5 mmol). To this mixture, chloroacetyl chloride (0.29 g, 2.60 mmol) dissolved in diethyl ether (5 mL) is added dropwise at room temperature. This mixture is stirred for 4 hours at room temperature. The mixture is partitioned between water (20 mL) and ether (20 mL). The organic layer is separated, dried (MgSO4) and concentrated. The residue is subjected to chromatography by elution with 10% ethyl acetate in heptane to give the desired product as a yellow solid (0.31 g). Physical characteristics: MS (ES−) for m/z 286 (M−H)⁺.

### Example 4

Preparation of 7:

Compound 3b (250 mg, 0.87 mmol) is dissolved in pyridine (5 mL) and treated with piperidine (87 mg, 0.99 mmol). The mixture is heated to 80° C. for 2 hours. After pyridine is removed the residue is chromatographed on a silica plate by elution with 10% ethyl acetate in heptane. The desired compound is isolated as a pale yellow solid (70 mg, 61% yield). Physical characteristics: MS (ES+) for m/z 337 (M+H)⁺; ¹H NMR (CDCl₃) δ 12.1, 3.90, 3.19, 2.78, 2.66, 2.54, 1.5-1.8.

Preparation of 2c:

To n-propyl cyanoacetate (2.54 g, 20 mmol) and sulfur (0.64 g) in DMF (4 mL), diethylamine (1.6 mL) is added under stirring. To this solution cyclohexanone (2.03 g, 20 mmol) is added dropwise. The mixture is heated to 60° C. for 2 hours and it is poured into water (30 mL), which is extracted with diethyl ether (30 mL). The ethereal solution is dried with sodium sulfate and concentrated to give the desired compound as an orange solid (2.85 g). Physical characteristics: MS (ES+) for m/z 240.

Preparation of 3c from 2c:

Compound 2c (2.50 g, 10.5 mmol) is dissolved in diethyl ether (45 mL) and treated with triethyl amine (1.8 mL, 12.5 mmol). To this mixture, chloroacetyl chloride (1.28 g, 11.5 mmol) dissolved in diethyl ether (5 mL) is added dropwise at room temperature. This mixture is stirred for 4 hours at room temperature. The mixture is partitioned between water (80 mL) and ether (80 mL). The organic layer is separated, dried (MgSO4) and concentrated. The residue is subjected to chromatography by elution with 50% diethyl ether in heptane to give the desired product as a green solid (1.8 g). Physical characteristics: MS (ES−) for m/z 314 (M−H)⁺.

### Example 5

Preparation of 8:

Compound 3c (1.8 g, 5.74 mmol) is dissolved in pyridine (25 mL) and treated with piperidine (0.57 g, 6.53 mmol). The mixture is heated to 80° C. for 2 hours. After pyridine is removed the residue is chromatographed on a silica plate by elution with 10% ethyl acetate in heptane. The desired compound is isolated as a white solid (0.23 g). Physical characteristics: MS (ES+) for m/z 365 (M+H)⁺; ¹H NMR (CDCl₃) δ 12.2, 4.28, 3.19, 2.80, 2.67, 2.53, 1.5-1.8, 1.05.

Preparation of 2d:

To i-propyl cyanoacetate (2.54 g, 20 mmol) and sulfur (0.64 g) in DMF (4 mL), diethylamine (1.6 mL) is added under stirring. To this solution cyclohexanone (2.03 g, 20 mmol) is added dropwise. The mixture is heated to 60° C. for 2 hours and it is poured into water (30 mL), which is extracted with diethyl ether (30 mL). The ethereal solution is dried with sodium sulfate and concentrated to give the desired compound as an orange solid (1.43 g). Physical characteristics: MS (ES+) for m/z 240.

Preparation of 3d from 2d:

Compound 2d (1 g, 4.2 mmol) is dissolved in diethyl ether (18 mL) and treated with triethyl amine (0.7 mL, 5.7 mmol). To this mixture, chloroacetyl chloride (0.51 g, 4.6 mmol) dissolved in diethyl ether (5 mL) is added dropwise at room temperature. This mixture is stirred for 4 hours at room temperature. The mixture is partitioned between water (80 mL) and ether (80 mL). The organic layer is separated, dried (MgSO4) and concentrated. The residue is triturated with heptane and the precipitate is collected as the desired product (0.35 g, tan solid). Physical characteristics: MS (ES−) for m/z 314 (M−H)⁺.

### Example 6

Preparation of 9:

Compound 3d (0.286 g, 0.91 mmol) is dissolved in pyridine (4 mL) and treated with piperidine (90 mg, 1.04 mmol). The mixture is heated to 80° C. for 2 hours. After pyridine is removed the residue is chromatographed on a silica plate by elution with 10% ethyl acetate in heptane. The desired compound is isolated as a white solid (93 mg). Physical characteristics: MS (ES+) for m/z 365 (M+H)⁺; ¹H NMR (CDCl₃) δ 12.2, 5.29, 3.19, 2.79, 2.67, 2.53, 1.4-1.9, 1.37.

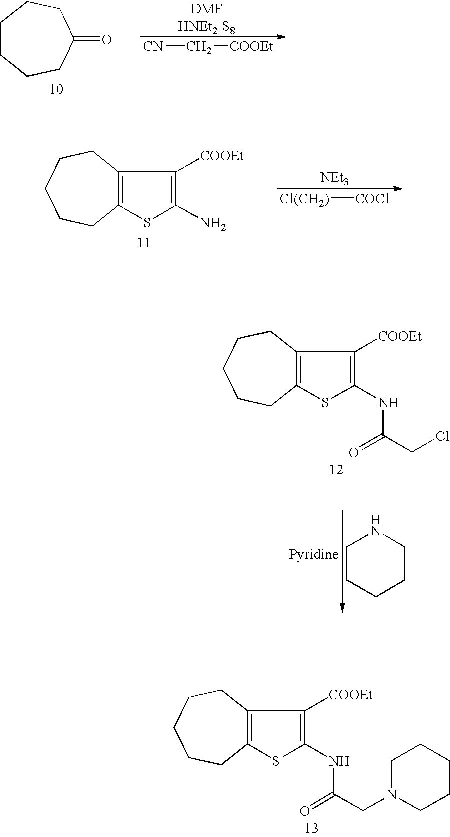

Preparation of 11:

To ethyl cyanoacetate (4.0 g, 35 mmol) and sulfur (1.12 g) in DMF (7 mL), diethylamine (2.8 mL) is added under stirring. To this solution cycloheptanone (3.94 g, 35 mmol) is added dropwise. The mixture is heated to 60° C. for 2 hours and it is poured into water (60 mL), which is extracted with diethyl ether (50 mL). The ethereal solution is dried with sodium sulfate and concentrated. The residue is subjected to chromatography by elution with 25% ethyl acetate in heptane to give the desired compound as a light yellow solid (3.48 g, 42% yield). Physical characteristics: MS (ES+) for m/z 240.

Preparation of 12:

Compound 11 (2.0 g, 8.36 mmol) is dissolved in diethyl ether (60 mL) and treated with triethyl amine (1.3 mL, 9.2 mmol). To this mixture, chloroacetyl chloride (1.04 g, 9.2 mmol) dissolved in diethyl ether (5 mL) is added dropwise at room temperature. This mixture is stirred for 4 hours at room temperature. The mixture is partitioned between water (30 mL) and ether (30 mL). The organic layer is separated, dried (MgSO4) and concentrated. The residue is subjected to chromatography by elution with 5% ethyl acetate in heptane to give the desired product as a white solid (0.965 g, 37% yield). Physical characteristics: MS (ES−) for m/z 314 (M−H)⁺.

### Example 7

Preparation of 13:

Compound 12 (0.3 g, 0.95 mmol) is dissolved in pyridine (6 mL) and treated with piperidine (98 mg, 1.14 mmol). The mixture is heated to 80° C. for 2 hours. After pyridine is removed the residue is chromatographed on a silica plate by elution with 10% ethyl acetate in heptane. The desired compound is isolated as a white solid (0.20 g, 35% yield). Physical characteristics: MS (ES+) for m/z 365 (M+H)⁺; ¹H NMR (CDCl₃) δ 12.1, 4.40, 3.18, 3.05, 2.74, 2.53, 1.5-1.9, 1.41.

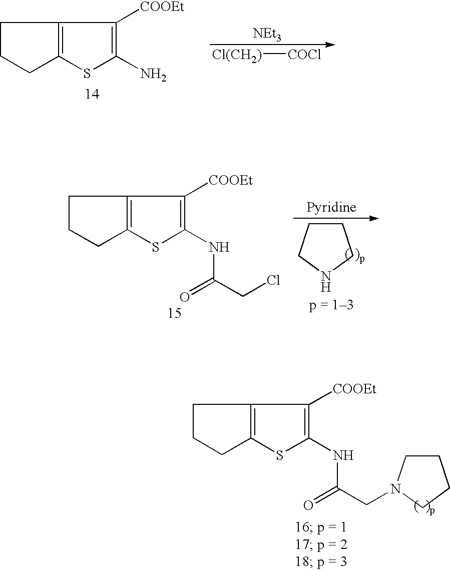

Preparation of 15 from 14:

Compound 14 (2.3 g, 10.9 mmol) is dissolved in diethyl ether (60 mL) and treated with triethyl amine (2.9 mL, 21.8 mmol). To this mixture, chloroacetyl chloride (1.46 g, 21.8 mmol) dissolved in diethyl ether (5 mL) is added dropwise at room temperature. This mixture is stirred for 4 hours at room temperature. Insoluble material is filtered off and the filtrate is concentrated. The residue is triturated with ethanol (30 mL) and the precipitate is collected, washed with ethanol (2×10 mL) and dried to give a tan solid (2.0 g, 61% yield). Physical characteristics: MS (ES−) for m/z 286, 288 (M+H)⁺.

### Example 8

Preparation of 16:

Compound 14 (70 mg, 0.23 mmol) is dissolved in pyridine (2 mL) and treated with pyrrolidine (43 mg, 0.6 mmol). The mixture is heated to 80° C. for 2 hours. After pyridine is removed the residue is chromatographed on a silica plate by elution with 20% ethyl acetate in hexanes. The desired compound is isolated as a white solid (57 mg, 77% yield). Physical characteristics: MS (ES+) for m/z 323 (M+H)⁺; ¹H NMR (CDCl₃) δ 12.0, 4.31, 3.39, 2.90, 2.84, 2.71, 2.37, 1.89, 1.38.

### Example 9

Preparation of 17:

Compound 14 (50 mg, 0.16 mmol) is dissolved in pyridine (2 mL) and treated with piperidine (43 mg, 0.5 mmol). The mixture is heated to 80° C. for 2 hours. After pyridine is removed the residue is chromatographed on a silica plate by elution with 20% ethyl acetate in hexanes. The desired compound is isolated as a white solid (30 mg, 56% yield). Physical characteristics: MS (ES+) for m/z 337 (M+H)⁺; ¹H NMR (CDCl₃) δ 4.35, 3.21, 2.91, 2.86, 2.6, 2.39, 1.78, 1.54, 1.38.

### Example 10

Preparation of 18:

Compound 14 (70 mg, 0.23 mmol) is dissolved in pyridine (2 mL) and treated with hexamethyleneimine (43 mg, 0.44 mmol). The mixture is heated to 80° C. for 2 hours. After pyridine is removed the residue is chromatographed on a silica plate by elution with 20% ethyl acetate in hexanes. The desired compound is isolated as a yellow solid (76 mg, 947% yield). Physical characteristics: MS (ES+) for m/z 351 (M+H)⁺; ¹H NMR (CDCl₃) δ 12.1, 4.32, 3.35, 2.90, 2.84, 2.76, 2.37, 1.6-1.8, 1.36.

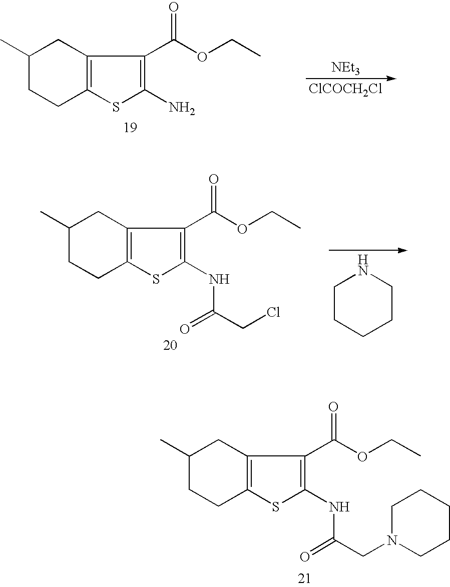

Preparation of 20 from 19:

Compound 15 (50 mg, 0.21 mmol) is dissolved in methylene chloride (10 mL) and treated with triethyl amine (0.13 mL, 1 mmol). To this mixture, chloroacetyl chloride (0.08 g, 1 mmol) dissolved in diethyl ether (1 mL) is added dropwise at room temperature. This mixture is stirred for 4 hours at room temperature. Insoluble material is filtered off and the filtrate is concentrated. The mixture is partitioned between water (20 mL) and ether (20 mL). The organic layer is separated, dried (MgSO4) and concentrated. The residue is subjected to chromatography by elution with 20% ethyl acetate in hexanes to give the desired product as a yellow oil (50 mg, 76% yield). Physical characteristics: MS (ES−) for m/z 314 (M−H)⁺.

### Example 11

Preparation of 21:

Compound 20 (50 mg, 0.16 mmol) is dissolved in pyridine (2 mL) and treated with piperidine (43 mg, 0.5 mmol). The mixture is heated to 80° C. for 2 hours. After pyridine is removed the residue is chromatographed on a silica plate by elution with 20% ethyl acetate in hexanes. The desired compound is isolated as a yellow oil (37 mg, 64% yield). Physical characteristics: MS (ES+) for m/z 365 (M+H)⁺; ¹H NMR (CDCl₃) δ12.2, 4.4, 3.71, 2.97, 2.70, 2.52, 2.26, 1.8-1.9, 1.70, 1.5, 1.4, 1.05.

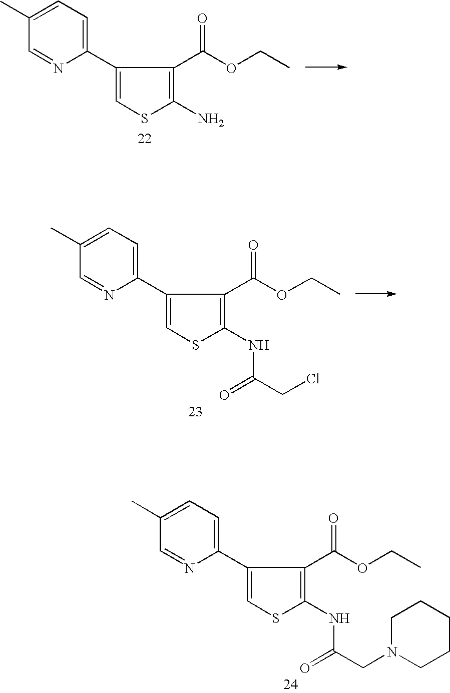

Preparation of 23 from 22:

Compound 22 (40 mg, 0.15 mmol) is dissolved in methylene chloride (10 mL) and treated with triethyl amine (0.067 mL, 0.5 mmol). To this mixture, chloroacetyl chloride (0.04 g, 0.5 mmol) dissolved in diethyl ether (1 mL) is added dropwise at room temperature. This mixture is stirred for 4 hours at room temperature. Insoluble material is filtered off and the filtrate is concentrated. The mixture is partitioned between water (20 mL) and ether (20 mL). The organic layer is separated, dried (MgSO4) and concentrated. The residue is subjected to chromatography by elution with 33% ethyl acetate in hexanes to give the desired product as a yellow solid (30 mg, 59% yield). Physical characteristics: MS (ES−) for m/z 337 (M−H)⁺.

### Example 12

Preparation of 24:

Compound 23 (30 mg, 0.09 mmol) is dissolved in pyridine (2 mL) and treated with piperidine (17 mg, 0.2 mmol). The mixture is heated to 80° C. for 2 hours. After pyridine is removed the residue is chromatographed on a silica plate by elution with 33% ethyl acetate in hexanes. The desired compound is isolated as a yellow solid (7 mg, 20% yield). Physical characteristics: MS (ES+) for m/z 388 (M+H)⁺; ¹H NMR (CDCl₃) δ 12.3, 8.44, 7.52, 7.13, 4.15, 3.23, 2.59, 2.56, 1.71, 1.50, 0.99.

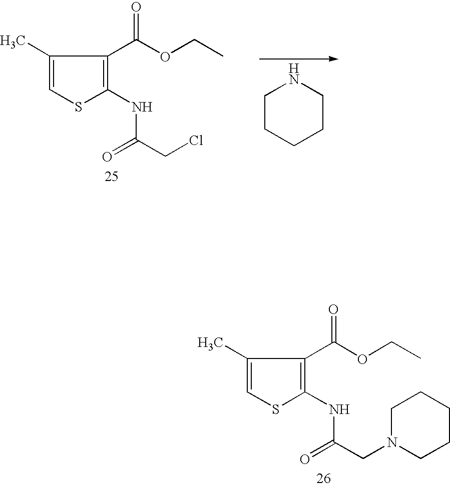

### Example 13

Compound 23 (100 mg, 0.38 mmol) is dissolved in pyridine (2 mL) and treated with piperidine (68 mg, 0.8 mmol). The mixture is heated to 80° C. for 2 hours. After pyridine is removed the residue is chromatographed on a silica plate by elution with 20% ethyl acetate in hexanes. The desired compound is isolated as a white solid (90 mg, 76% yield). Physical characteristics: MS (ES+) for m/z 311 (M+H)⁺; ¹H NMR (CDCl₃) δ 12.3, 6.39, 4.39, 3.19, 2.53, 2.37, 1.71, 1.48, 1.40.

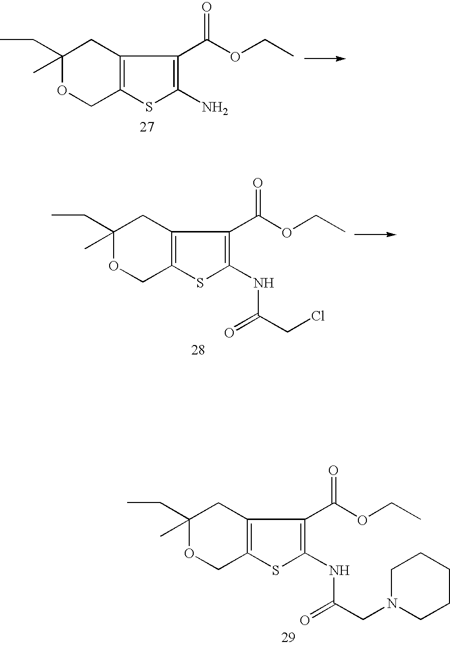

Preparation of 28 from 27:

Compound 27 (35 mg, 0.13 mmol) is dissolved in methylene chloride (10 mL) and treated with triethyl amine (0.067 mL, 0.5 mmol). To this mixture, chloroacetyl chloride (0.04 g, 0.5 mmol) dissolved in diethyl ether (1 mL) is added dropwise at room temperature. This mixture is stirred for 4 hours at room temperature. Insoluble material is filtered off and the filtrate is concentrated. The mixture is partitioned between water (20 mL) and ethyl acetate (20 mL). The organic layer is separated, dried (MgSO4) and concentrated. The residue is subjected to chromatography by elution with 20% ethyl acetate in hexanes to give the desired product as a yellow oil (9 mg, 20% yield). Physical characteristics: MS (ES−) for m/z 344, 346 (M−H)⁺.

### Example 14

Compound 23 (9 mg, 0.03 mmol) is dissolved in pyridine (2 mL) and treated with piperidine (9 mg, 0.1 mmol). The mixture is heated to 80° C. for 2 hours. After pyridine is removed the residue is chromatographed on a silica plate by elution with 20% ethyl acetate in hexanes. The desired compound is isolated as a yellow oil. (7 mg, 70% yield). Physical characteristics: MS (ES+) for m/z 395 (M+H)⁺; ¹H NMR (CDCl₃) δ 12.23, 4.68, 4.38, 3.19, 2.70, 2.53, 1.70, 1.53, 1.39, 0.97.

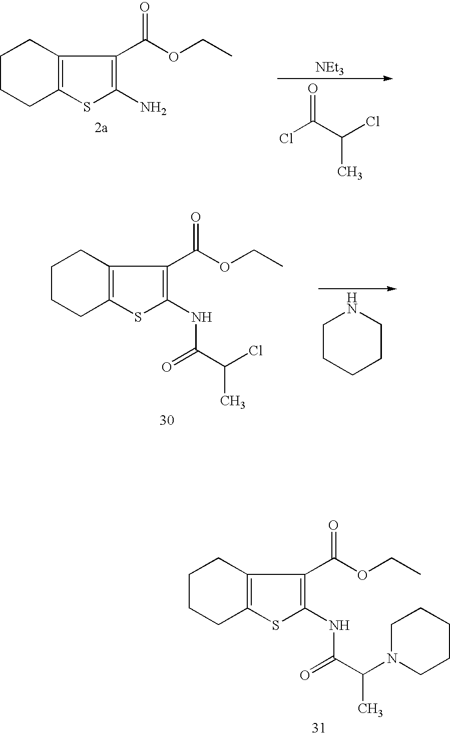

Preparation of 30 from 2a:

Compound 2a (45 mg, 0.2 mmol) is dissolved in methylene chloride (2 mL) and treated with triethyl amine (0.1 mL, 0.7 mmol). To this mixture, 2-chloropropionyl chloride (0.058 mL, 0.6 mmol) dissolved in diethyl ether (1 mL) is added dropwise at room temperature. This mixture is stirred for 4 hours at room temperature. Insoluble material is filtered off and the filtrate is concentrated. The mixture is partitioned between water (20 mL) and methylene chloride (20 mL). The organic layer is separated, dried (MgSO4) and concentrated. The residue is subjected to chromatography by elution with 15% ethyl acetate in hexanes to give the desired product as a white solid (40 mg, 63% yield). Physical characteristics: MS (ES−) for m/z 314, 316 (M−H)⁺.

### Example 15

Preparation of 31:

Compound 30 (30 mg, 0.095 mmol) is dissolved in pyridine (2 mL) and treated with piperidine (20 mg, 0.24 mmol). The mixture is heated to 80° C. for 2 hours. After pyridine is removed the residue is chromatographed on a silica plate by elution with 15% ethyl acetate in hexanes. The desired compound is isolated as a white semi-solid (6 mg, 17% yield). Physical characteristics: MS (ES+) for m/z 365 (M+H)⁺; ¹H NMR (CDCl₃) δ 12.33, 4.39, 3.35, 3.19, 2.80, 2.67, 2.4-2.6, 1.6-1.9, 1.40, 1.30.

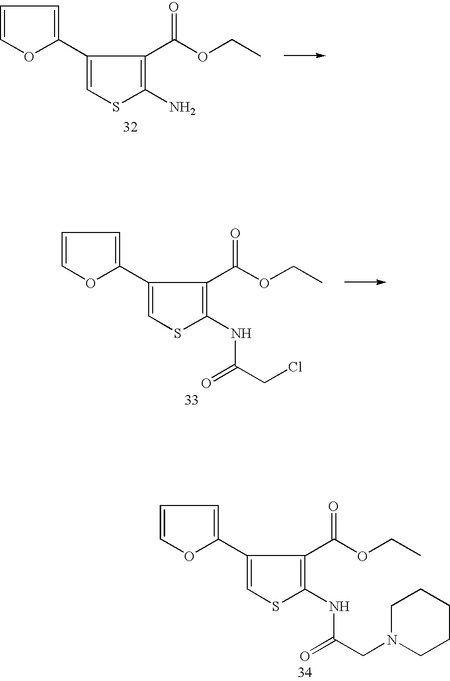

Preparation of 33 from 32:

Compound 32 (40 mg, 0.17 mmol) is dissolved in methylene chloride (10 mL) and treated with triethyl amine (0.067 mL, 0.5 mmol). To this mixture, chloroacetyl chloride (0.04 g, 0.5 mmol) dissolved in diethyl ether (1 mL) is added dropwise at room temperature. This mixture is stirred for 4 hours at room temperature. Insoluble material is filtered off and the filtrate is concentrated. The mixture is partitioned between water (20 mL) and methylene chloride (20 mL). The organic layer is separated, dried (MgSO₄) and concentrated. The residue is subjected to chromatography by elution with 20% ethyl acetate in hexanes to give the desired product as a white solid (12 mg, 23% yield). Physical characteristics: MS (ES−) for m/z 312, 314 (M−H)⁺.

### Example 16

Preparation of 34:

Compound 33 (12 mg, 0.04 mmol) is dissolved in pyridine (2 mL) and treated with piperidine (9 mg, 0.1 mmol). The mixture is heated to 80° C. for 2 hours. After pyridine is removed the residue is chromatographed on a silica plate by elution with 20% ethyl acetate in hexanes. The desired compound is isolated as a white solid (12 mg, 87% yield). Physical characteristics: MS (ES+) for m/z 363 (M+H)⁺; ¹H NMR (CDCl₃) δ 12.25, 7.43, 6.90, 6.43, 4.28, 3.21, 2.55, 1.72, 1.50, 1.21.

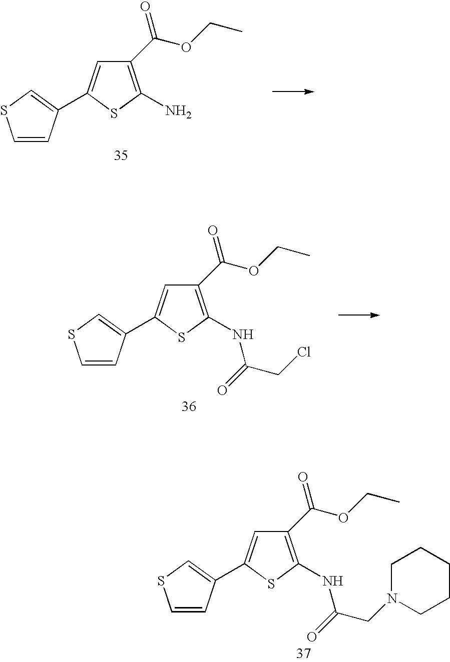

Preparation of 36 from 35:

Compound 35 (45 mg, 0.18 mmol) is dissolved in diethyl ether (2 mL) and treated with triethyl amine (0.075 mL, 0.56 mmol). To this mixture, chloroacetyl chloride (0.043 mL, 0.54 mmol) dissolved in diethyl ether (1 mL) is added dropwise at room temperature. This mixture is stirred for 1 hour at room temperature. Insoluble material is filtered off and the filtrate is concentrated. The mixture is partitioned between water (20 mL) and diethyl ether (20 mL). The organic layer is separated, dried (MgSO₄) and concentrated. The residue is subjected to chromatography by elution with 17% ethyl acetate in hexanes to give the desired product as a white solid (53 mg, 90% yield). Physical characteristics: MS (ES−) for m/z 328, 330 (M−H)⁺.

### Example 17

Preparation of 37:

Compound 37 (40 mg, 0.12 mmol) is dissolved in pyridine (2 mL) and treated with piperidine (31 mg, 0.36 mmol). The mixture is heated to 80° C. for 2 hours. After pyridine is removed the residue is chromatographed on a silica plate by elution with 17% ethyl acetate in hexanes. The desired compound is isolated as a white solid (44 mg, 96% yield). Physical characteristics: MS (ES+) for m/z 379 (M+H)⁺;¹ H NMR (CDCl₃) δ 12.3, 7.29, 7.02, 6.78, 6.43, 4.22, 3.24, 2.57, 1.74, 1.51, 1.11.

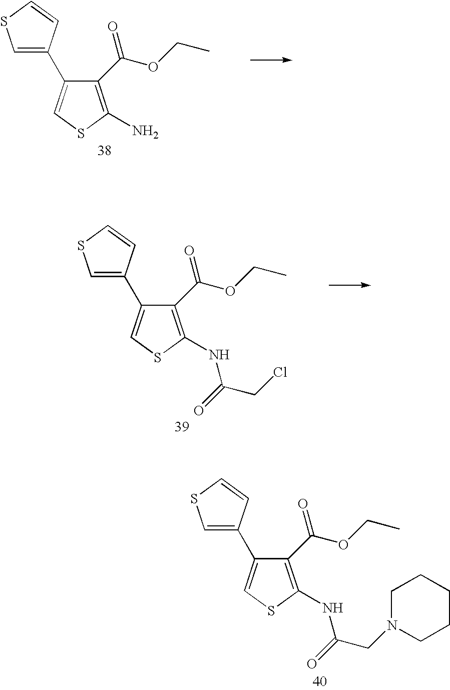

Preparation of 39 from 38:

Compound 38 (43 mg, 0.17 mmol) is dissolved in methylene chloride (4 mL) and treated with triethyl amine (0.07 mL, 0.51 mmol). To this mixture, chloroacetyl chloride (0.04 mL, 0.51 mmol) dissolved in diethyl ether (1 mL) is added dropwise at room temperature. This mixture is stirred for 1 hour at room temperature. Insoluble material is filtered off and the filtrate is concentrated. The mixture is partitioned between water (20 mL) and methylene chloride (20 mL). The organic layer is separated, dried (MgSO₄) and concentrated. The residue is subjected to chromatography by elution with 17% ethyl acetate in hexanes to give the desired product as a white solid (49 mg, 88% yield). Physical characteristics: MS (ES−) for m/z 328, 330 (M−H)⁺.

### Example 18

Preparation of 40:

Compound 39 (22 mg, 0.067 mmol) is dissolved in pyridine (2 mL) and treated with piperidine (17 mg, 0.2 mmol). The mixture is heated to 80° C. for 2 hours. After pyridine is removed the residue is chromatographed on a silica plate by elution with 17% ethyl acetate in hexanes. The desired compound is isolated as a white solid (23 mg, 92% yield). Physical characteristics: MS (ES+) for m/z 379 (M+H)⁺; ¹H NMR (CDCl₃) δ 12.3, 7.29, 7.02, 6.78, 6.43, 4.22, 3.24, 2.57, 1.74, 1.51, 1.11.

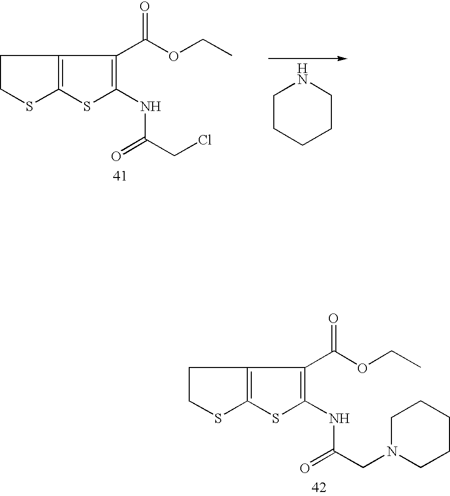

### Example 19

Preparation of 42:

Compound 41 (30 mg, 0.1 mmol) is dissolved in pyridine (1 mL) and treated with piperidine (17 mg, 0.2 mmol). The mixture is heated to 80° C. for 2 hours. After pyridine is removed the residue is chromatographed on a silica plate by elution with 20% ethyl acetate in hexanes. The desired compound is isolated as a white solid (20 mg, 59% yield). Physical characteristics: MS (ES+) for m/z 355 (M+H)⁺; ¹H NMR (CDCl₃) δ 12.1, 4.36, 4.23, 4.13, 3.91, 3.28, 3.18, 2.53, 1.71, 1.49, 1.37.

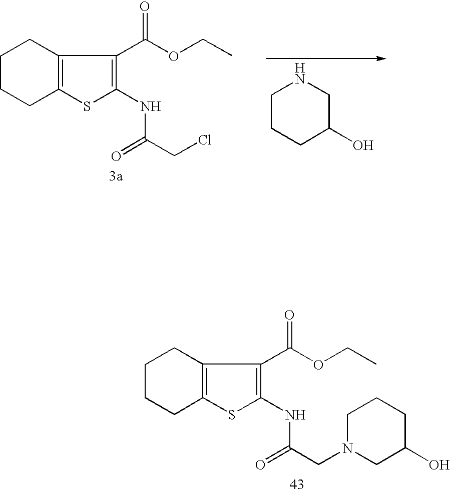

### Example 20

Preparation of 43:

Compound 3a (30 mg, 0.1 mmol) is dissolved in pyridine (1 mL) and treated with 3-hydroxypiperidine (20 mg, 0.2 mmol). The mixture is heated to 80° C. for 2 hours. After pyridine is removed the residue is chromatographed on a silica plate by elution with ethyl acetate. The desired compound is isolated as a white solid (26 mg, 70% yield). Physical characteristics: MS (ES+) for m/z 367 (M+H)⁺; ¹H NMR (CDCl₃) δ 12.6, 4.35, 3.92, 3.64, 3.19, 2.5-2.9, 2.33, 2.02, 1.791.4-1.7, 1.37.

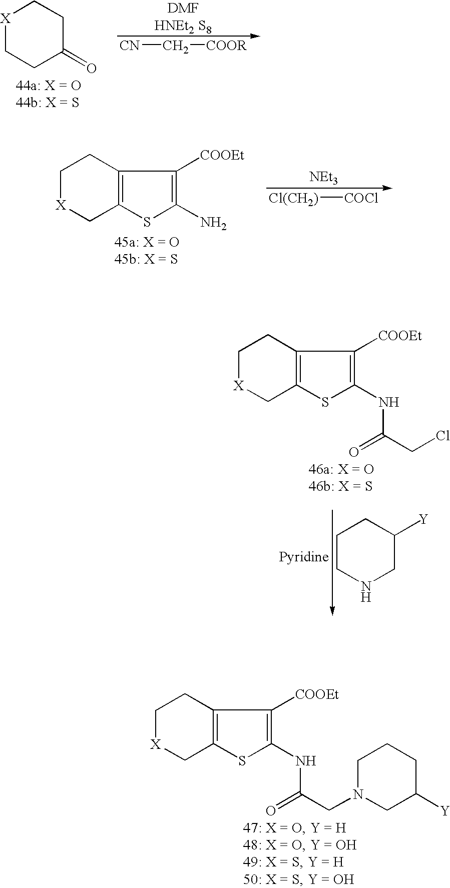

Preparation of 45a from 44a:

To ethyl cyanoacetate (226 mg, 2 mmol) and sulfur (76 mg) in DMF (2 mL), triethylamine (0.25 mL) is added under stirring. To this solution tetrahydro-4H-pyran-4-one (44a, 200 mg, 2 mmol) is added dropwise. The mixture is stirred at room temperature for 16 hours and it is poured into water (20 mL), which is extracted with diethyl ether (20 mL). The ethereal solution is dried with sodium sulfate and concentrated. The residue is triturated with hexanes (10 mL) and the precipitate is collected to give a white solid (0.2 g, 44% yield). Physical characteristics: MS (ES+) for m/z 228 (M+H)⁺.

Preparation of 45b from 44b:

To ethyl cyanoacetate (226 mg, 2 mmol) and sulfur (76 mg) in DMF (2 mL), triethylamine (0.25 mL) is added under stirring. To this solution tetrahydrothiopyran-4-one (44b, 232 mg, 2 mmol) is added. The mixture is stirred at room temperature for 16 hours and it is poured into water (20 mL), which is extracted with diethyl ether (20 mL). The ethereal solution is dried with sodium sulfate and concentrated. The residue is triturated with hexanes (10 mL) and the precipitate is collected to give a yellow solid (0.33 g, 68% yield). Physical characteristics: MS (ES+) for m/z 244 (M+H)⁺.

Preparation of 46a from 45a:

Compound 45a (174 mg, 0.77 mmol) is dissolved in methylene chloride (10 mL) and treated with triethyl amine (0.4 mL, 3.1 mmol). To this mixture, chloroacetyl chloride (0.18 mL, 2.3 mmol) dissolved in diethyl ether (1 mL) is added dropwise at room temperature. This mixture is stirred for 4 hours at room temperature. The mixture is partitioned between water (10 mL) and ether (10 mL). The organic layer is separated, dried (MgSO4) and concentrated. The residue is subjected to chromatography by elution with 20% ethyl acetate in hexane to give the desired product as a white solid (130 mg). Physical characteristics: MS (ES−) for m/z 302, 304 (M−H)⁺.

Preparation of 46b from 45b:

Compound 45b (243 mg, 1 mmol) is dissolved in methylene chloride (10 mL) and treated with triethyl amine (0.55 mL, 4 mmol). To this mixture, chloroacetyl chloride (0.24 mL, 3 mmol) dissolved in diethyl ether (1 mL) is added dropwise at room temperature. This mixture is stirred for 2 hours at room temperature. The mixture is partitioned between water (10 mL) and methylene chloride (10 mL). The organic layer is separated, dried (MgSO₄) and concentrated. The residue is triturated with methanol and the precipitate is collected as the desired product (0.23 g, tan solid). Physical characteristics: MS (ES+) for m/z 320, 322 (M+H)⁺.

### Example 21

Preparation of 47:

Compound 46a (30 mg, 0.1 mmol) is dissolved in pyridine (1 mL) and treated with piperidine (17 mg, 0.2 mmol). The mixture is heated to 80° C. for 2 hours. After pyridine is removed the residue is chromatographed on a silica plate by elution with 20% ethyl acetate in hexanes. The desired compound is isolated as a white solid (28 mg, 80% yield). Physical characteristics: MS (ES+) for m/z 353 (M+H)⁺; ¹H NMR (CDCl₃) δ 12.3, 4.71, 4.36, 3.94, 3.19, 2.90, 1.71, 1.49, 1.37.

### Example 22

Preparation of 48:

Compound 46a (30 mg, 0.1 mmol) is dissolved in pyridine (1 mL) and treated with 3-hydroxypiperidine (20 mg, 0.2 mmol). The mixture is heated to 80° C. for 2 hours. After pyridine is removed the residue is chromatographed on a silica plate by elution with 5% methanol in methylene chloride. The desired compound is isolated as a white solid (28 mg, 76% yield). Physical characteristics: MS (ES+) for m/z 369 (M+H)+: ¹H NMR (CDCl) δ 12.6, 4.71. 4.36, 3.94, 3.21. 2.88, 1.5-2.8. 1.37.

### Example 23

Preparation of 49:

Compound 46b (32 mg, 0.1 mmol) is dissolved in pyridine (1 mL) and treated with piperidine (17 mg, 0.2 mmol). The mixture is heated to 80° C. for 2 hours. After pyridine is removed the residue is chromatographed on a silica plate by elution with 20% ethyl acetate in hexanes. The desired compound is isolated as a white solid (31 mg, 84% yield). Physical characteristics: MS (ES+) for m/z 369 (M+H)⁺; ¹H NMR (CDCl₃) δ 12.3, 4.37, 3.72, 3.18, 3.10, 2.91, 2.52, 1.70, 1.49, 1.38.

### Example 24

Preparation of 50:

Compound 46b (32 mg, 0.1 mmol) is dissolved in pyridine (1 mL) and treated with 3-hydroxypiperidine (20 mg, 0.2 mmol). The mixture is heated to 80° C. for 2 hours. After pyridine is removed the residue is chromatographed on a silica plate by elution with 50% ethyl acetate in hexanes. The desired compound is isolated as a white solid (28 mg, 74% yield). Physical characteristics: MS (ES+) for m/z 385 (M+H)⁺; ¹H NMR (CDCl₃) δ 12.7, 4.37, 3.93, 3.73, 3.55, 3.21, 3.07, 2.91, 1.5-2.9, 1.37.

Compounds of this invention prepared following the above procedures but making non-critical variations include:

a) ethyl 2-{[2-(1-piperidinyl)acetyl]amino}-5,6-dihydro-4H-cyclopenta[b]thiophene-3-carboxylate;

b) ethyl 2-[(morpholin-4-ylacetyl)amino]-5,6-dihydro-4H-cyclopenta[b]thiophene-3-carboxylate;

c) ethyl 2-[(piperidin-1-ylacetyl)amino]-4,5,6,7-tetrahydro-1-benzothiophene-3-carboxylate;

d) ethyl 2-[(piperidin-1-ylacetyl)amino]-4,5,6,7,8,8a-hexahydro-3aH-cyclohepta[b]thiophene-3-carboxylate;

e) ethyl 2-[(morpholin-4-ylacetyl)amino]-4,5,6,7,8,8a-hexahydro-3aH-cyclohepta[b]thiophene-3-carboxylate;

f) ethyl 2-{[(4-phenylpiperidin- 1-yl)acetyl]amino}-5,6-dihydro-4H-cyclopenta[b]thiophene-3-carboxylate;

g) ethyl 2-[(pyrrolidin-1-ylacetyl)amino]-4,5,6,7-tetrahydro-1-benzothiophene-3-carboxylate;

h) ethyl 2-[(azepan-1-ylacetyl)amino]-4,5,6,7-tetrahydro-1-benzothiophene-3-carboxylate;

i) ethyl 2-[(azetidin-1-ylacetyl)amino]-4,5,6,7-tetrahydro-1-benzothiophene-3-carboxylate;

j) ethyl 2-[(pyrrolidin-1-ylacetyl)amino]-5,6-dihydro-4H-cyclopenta[b]thiophene-3-carboxylate;

k) ethyl 2-[(azepan-1-ylacetyl)amino]-5,6-dihydro-4H-cyclopenta[b]thiophene-3-carboxylate;

l) ethyl 5-chloro-4-phenyl-2-[(piperidin-1-ylacetyl)amino]thiophene-3-carboxylate;

m) ethyl 6-phenyl-2-[(piperidin-1-ylacetyl)amino]-4,5,6,7-tetrahydro-1-benzothiophene-3-carboxylate;

n) ethyl 6-tert-butyl-2-[(piperidin-1-ylacetyl)amino]-4,5,6,7-tetrahydro-1-benzothiophene-3-carboxylate;

o) ethyl 2-[(2-piperidin-1-ylpropanoyl)amino]-4,5,6,7-tetrahydro-1-benzothiophene-3-carboxylate;

p) diethyl 2-[(piperidin-1-ylacetyl)amino]-4,7-dihydrothieno[2,3-c]pyridine-3,6(5H)-dicarboxylate;

q) ethyl 5-methyl-2-[(piperidin-1-ylacetyl)amino]-4,5,6,7-tetrahydro-1-benzothiophene-3-carboxylate;

r) ethyl 4-(5-methylpyridin-2-yl)-2-[(piperidin-1-ylacetyl)amino]thiophene-3-carboxylate;

s) ethyl 5-ethyl-5-methyl-2-[(piperidin-1-ylacetyl)amino]-4,7-dihydro-5H-thieno[2,3-c]pyran-3-carboxylate;

t) ethyl 4-(2-furyl)-2-[(piperidin-1-ylacetyl)amino]thiophene-3-carboxylate;

u) ethyl 6-benzyl-2-[(piperidin-1-ylacetyl)amino]-4,5,6,7-tetrahydrothieno[2,3-c]pyridine-3-carboxylate;

v) ethyl 2-[(piperidin-1-ylacetyl)amino]-4-pyridin-4-ylthiophene-3-carboxylate;

w) ethyl 4-methyl-2-[(piperidin-1-ylacetyl)amino]thiophene-3-carboxylate;

x) methyl 2-[(piperidin-1-ylacetyl)amino]-4,5,6,7-tetrahydro-1-benzothiophene-3-carboxylate;

y) propyl 2-[(piperidin-1-ylacetyl)amino]-4,5,6,7-tetrahydro-1-benzothiophene-3-carboxylate;

z) ethyl 4-(4-bromophenyl)-2-[(piperidin-1-ylacetyl)amino]thiophene-3-carboxylate;

aa) ethyl 5-[(piperidin-1-ylacetyl)amino]-2,3′-bithiophene-4-carboxylate;

bb) ethyl 5-[(piperidin-1-ylacetyl)amino]-3,3′-bithiophene-4-carboxylate;

cc) isopropyl 2-[(piperidin-1-ylacetyl)amino]-4,5,6,7-tetrahydro-1-benzothiophene-3-carboxylate;

dd) isopropyl 4-(3,4-dichlorophenyl)-2-[(piperidin-1-ylacetyl)amino]thiophene-3-carboxylate;

ee) ethyl 2-[(piperidin-1-ylacetyl)amino]-4,5-dihydrothieno[2,3-b]thiophene-3-carboxylate;

ff) ethyl 2-{[(4-hydroxypiperidin-1-yl)acetyl]amino}-4,5,6,7-tetrahydro-1-benzothiophene-3-carboxylate; and

gg) ethyl 2-{[(3-hydroxypiperidin-1-yl)acetyl]amino}-4,5,6,7-tetrahydro-1-benzothiophene-3-carboxylate.

### Example 25

Biological Activity of Selected Compounds

Selected compounds were evaluated for their anti-parasitic activity in a binding assay as described in U.S. Patent No. 5,859,188 (Geary, et.al., 1999). Results of the evaluations are given in the following Table wherein % inhibition means percent displacement of a radiolabelled ligand as described.

| | | Testing Result |
|---|---|---|
| | | % inhibition at |
| Compound | Structure | indicated concentration |
| A | 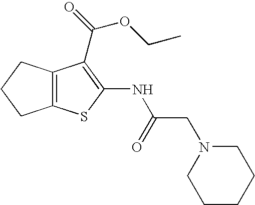 | 69% at 3 uM |
| B | 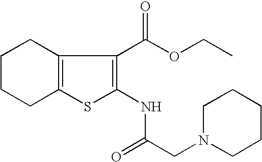 | 59% at 1 uM |
| C | 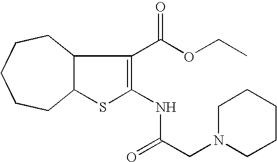 | Test 1: 82% at 25 uM Test 2: 53% |
| D | 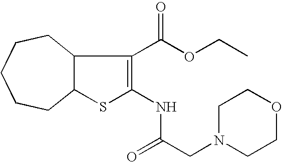 | 38% at 25 uM |
| E | 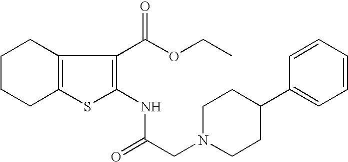 | 40% at 25 uM |
| f | 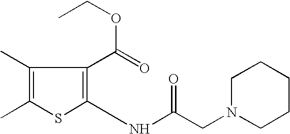 | 68% at 25 uM |
| g | 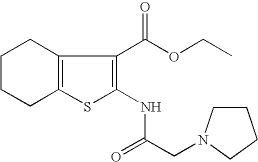 | 68-75% at 25 uM |
| h | 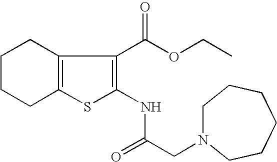 | 46% at 25 uM |
| i | 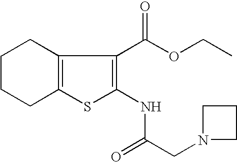 | 28% at 25 uM |
| j | 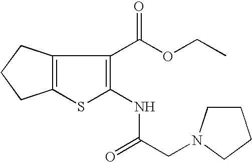 | 83-88% at 25 uM |
| k | 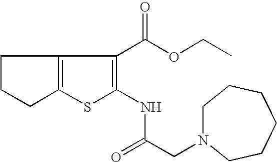 | 77-70% at 25 uM |
| l | 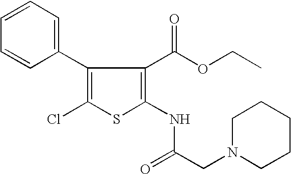 | 35% at 25 uM |
| m | 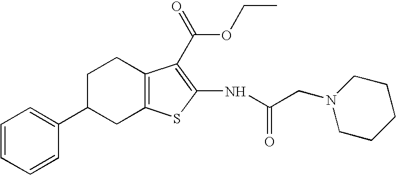 | 36% at 25 uM |
| n | 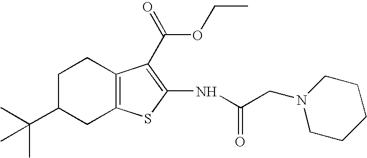 | 13% at 25 uM |
| o | 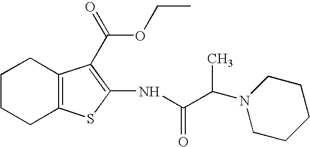 | Test 1: 46-73% at 25 uM Test 2: 38-40% |
| p | 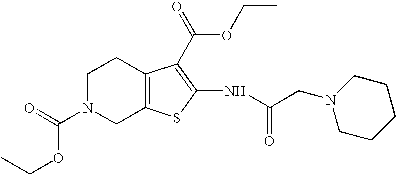 | Test 1: 50-52% at 25 uM Test 2: 33-49% |
| q | 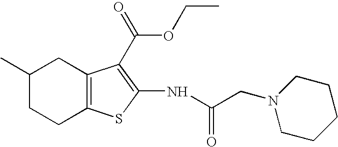 | Test 1: 65-74% at 25 uM Test 2: 15-22 |
| r | 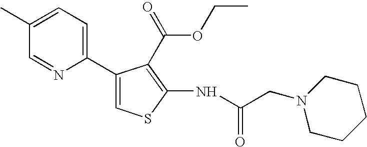 | Test 1: 42% at 25 uM Test 2: 26% |
| s | 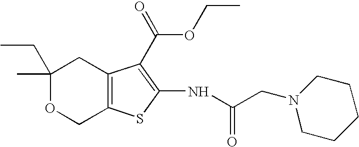 | Test 1: 93-100% at 25 uM Test 2: 69-79% |
| t | 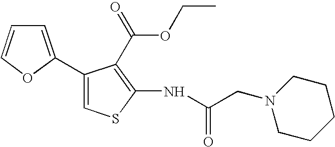 | Test 1: 70-75% at 25 uM Test 2: 24-40% |
| u | 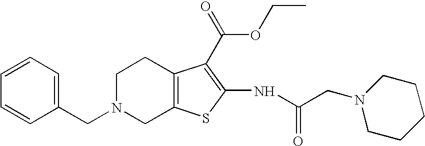 | Test 1: 48-51% at 25 uM Test 2: 0-14% |
| v | 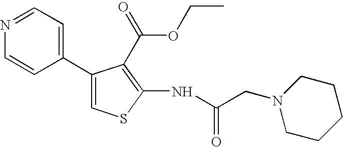 | Test 1: 15-43% at 25 uM Test 2: 14-17% |
| w | 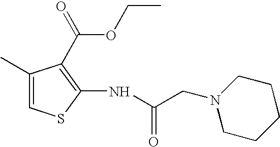 | Test 1: 58-70% at 25 uM Test 2: 39-47% |
| x | 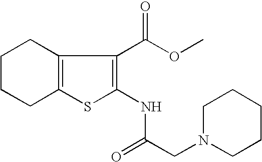 | Test 1: 55-70% at 25 uM Test 2: 41-55% |
| y | 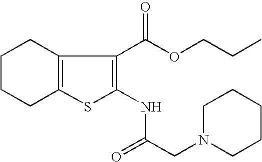 | Test 1: 52-65% at 25 uM Test 2: 33-54% |
| z | 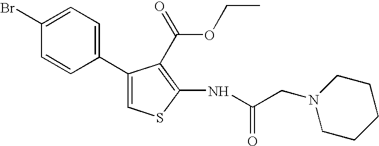 | Test 1: 44-55% at 25 uM Test 2: 37-43% |
| aa | 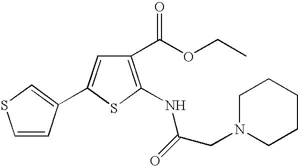 | Test 1: 46-66% at 25 uM Test 2: 37-43% |
| bb | 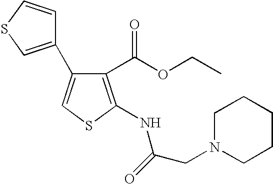 | Test 1: 40-63% at 25 uM Test 2: 18-38% |
| cc | 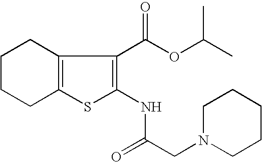 | 34-54% at 25 uM |
| dd | 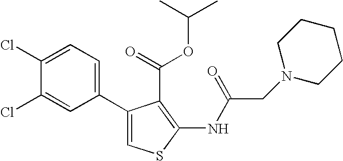 | 0-9% at 25 uM |
| ee | 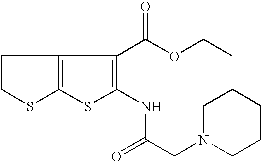 | 61-72% at 25 uM |
| ff | 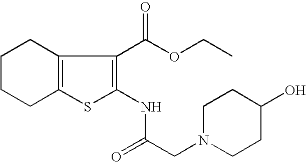 | 31-34% at 25 uM |
| gg | 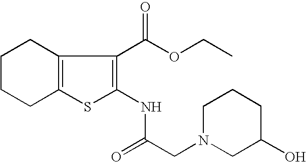 | 68-73% at 25 uM |

Having described the invention in detail and by reference to the preferred embodiments thereof, it will be apparent that modifications and variations are possible without departing from the scope of the appended claims.

What is claimed is:

1. A compound of Formula I comprising: 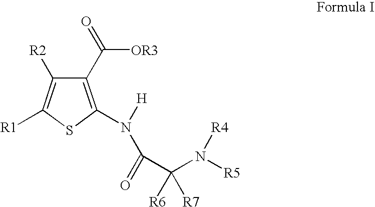
wherein R₁ and R₂ are selected from the group consisting of H, alkyl, phenyl, substituted phenyl, benzyl, substituted benzyl, heteroaryl, substituted heteroaryl, hetroarylmethylene, and substituted hetroarylmethylene; or
R₁ and R₂, along with the carbons to which they are attached, form a 5 to 7 membered substituted or unsubstituted carbocyclic or heterocyclic ring;
R₃ is alkyl of 1 to 4 carbons;
R₄, and R₅ are independently alkyl, heteroalkyl, cycloalkyl, aryl, aralkyl, heteroaryl or heteroaralkyl;
R₄ and R₅ taken together may form a 4-7 membered substituted or unsubstituted carbocyclic ring;
R₆ and R₇ are independently H or alkyl of 1 to 3 carbons.

2. A compound according to claim 1 selected from the group consisting of:
ethyl 2-{[(4-phenylpiperidin-1-yl)acetyl]amino}-5,6-dihydro-4H-cyclopenta[b]thiophene-3-carboxylate;
ethyl 2-[(azetidin-1-ylacetyl)amino]-4,5,6,7-tetrahydro-1-benzothiophene-3-carboxylate;
ethyl 4-(5-methylpyridin-2-yl)-2-[(piperidin-1-ylacetyl)amino]thiophene-3-carboxylate;
ethyl 5-ethyl-5-methyl-2-[(piperidin-1-ylacetyl)amino]-4,7-dihydro-5H-thieno[2,3-c]pyran-3-carboxylate;
ethyl 4-(2-furyl)-2-[(piperidin-1-ylacetyl)amino]thiophene-3-carboxylate;
ethyl 6-benzyl-2-[(piperidin-1-ylacetyl)amino]-4,5,6,7-tetrahydrothieno[2,3-c]pyridine-3-carboxylate;
ethyl 2-[(piperidin-1-ylacetyl)amino]-4-pyridin-4-ylthiophene-3-carboxylate;
ethyl 4-methyl-2-[(piperidin-1-ylacetyl)amino]thiophene-3-carboxylate;
ethyl 4-(4-bromophenyl)-2-[(piperidin-1-ylacetyl)amino]thiophene-3-carboxylate;
ethyl 5-[(piperidin-1-ylacetyl)amino]-2,3′-bithiophene-4-carboxylate;
ethyl 5-[(piperidin-1-ylacetyl)amino]-3,3′-bithiophene-4-carboxylate;
isopropyl 2-[(piperidin-1-ylacetyl)amino]-4,5,6,7-tetrahydro-1-benzothiophene-3-carboxylate;
Isopropyl 4-(3,4-dichlorophenyl)-2-[(piperidin-1-ylacetyl)amino]thiophene-3-carboxylate;
ethyl 2-[(piperidin-1-ylacetyl)amino]-4,5-dihydrothieno[2,3-b]thiophene-3-carboxylate;
ethyl 2-{[(4-hydroxypiperidin-1-yl)acetyl]amino}-4,5,6,7-tetrahydro-1-benzothiophene-3-carboxylate; and
ethyl 2-{[(3-hydroxypiperidin-1-yl)acetyl]amino}-4,5,6,7-tetrahydro-1-benzothiophene-3-carboxylate.

3. A method for treating or preventing parasitic diseases in mammals comprising the step of administering to the mammal an effective amount of a compound of claim 1.

4. A method for treating or preventing parasitic diseases in mammals according to claim 3 wherein the compound is selected from the group consisting of:
ethyl 2-{[2-( 1-piperidinyl)acetyl]amino}-5,6-dihydro-4H-cyclopenta[b]thiophene-3-carboxylate;
ethyl 2-[(morpholin-4-ylacetyl)amino]-5,6-dihydro-4H-cyclopenta[b]thiophene-3-carboxylate;
ethyl 2-[(piperidin-1-ylacetyl)amino]-4,5,6,7-tetrahydro-1-benzothiophene-3-carboxylate;
ethyl 2-[(piperidin-1-ylacetyl)amino]-4,5,6,7,8,8a-hexahydro-3aH-cyclohepta[b]thiophene-3-carboxylate;
ethyl 2-[(morpholin-4-ylacetyl)amino]-4,5,6,7,8,8a-hexahydro-3aH-cyclohepta[b]thiophene-3-carboxylate;
ethyl 2-{[(4-phenylpiperidin-1-yl)acetyl]amino}-5,6-dihydro-4H-cyclopenta[b]thiophene-3-carboxylate;
ethyl 2-[(pyrrolidin-1-ylacetyl)amino]-4,5,6,7-tetrahydro-1-benzothiophene-3-carboxylate;
ethyl 2-[(azepan-1-ylacetyl)amino]-4,5,6,7-tetrahydro-1-benzothiophene-3-carboxylate;
ethyl 2-[(azetidin-1-ylacetyl)amino]-4,5,6,7-tetrahydro-1-benzothiophene-3-carboxylate;
ethyl 2-[(pyrrolidin-1-ylacetyl)amino]-5,6-dihydro-4H-cyclopenta[b]thiophene-3-carboxylate;
ethyl 2-[(azepan-1-ylacetyl)amino]-5,6-dihydro-4H-cyclopenta[b]thiophene-3-carboxylate;
ethyl 5-chloro-4-phenyl-2-[(piperidin-1-ylacetyl)amino]thiophene-3-carboxylate;
ethyl 6-phenyl-2-[(piperidin-1-ylacetyl)amino]-4,5 ,6,7-tetrahydro-1-benzothiophene-3-carboxylate;
ethyl 6-tert-butyl-2-[(piperidin-1-ylacetyl)amino]-4,5,6,7-tetrahydro-1-benzothiophene-3-carboxylate;
ethyl 2-[(2-piperidin-1-ylpropanoyl)amino]-4,5,6,7-tetrahydro-1-benzothiophene-3-carboxylate;
diethyl 2-[(piperidin-1-ylacetyl)amino]-4,7-dihydrothieno[2,3-c]pyridine-3,6(5H)-dicarboxylate;
ethyl 5-methyl-2-[(piperidin-1-ylacetyl)amino]-4,5 ,6,7-tetrahydro-1-benzothiophene-3-carboxylate;
ethyl 4-(5-methylpyridin-2-yl)-2-[(piperidin-1-ylacetyl)amino]thiophene-3-carboxylate;
ethyl 5-ethyl-5-methyl-2-[(piperidin-1-ylacetyl)amino]-4,7-dihydro-5H-thieno[2,3-c]pyran-3-carboxylate;
ethyl 4-(2-furyl)-2-[(piperidin-1-ylacetyl)amino]thiophene-3-carboxylate;
ethyl 6-benzyl-2-[(piperidin-1-ylacetyl)amino]-4,5,6,7-tetrahydrothieno[2,3-c]pyridine-3-carboxylate;
ethyl 2-[(piperidin-1-ylacetyl)amino]-4-pyridin-4-ylthiophene-3-carboxylate;
ethyl 4-methyl-2-[(piperidin-1-ylacetyl)amino]thiophene-3-carboxylate;
methyl 2-[(piperidin-1-ylacetyl)amino]-4,5,6,7-tetrahydro-1-benzothiophene-3-carboxylate;
propyl 2-[(piperidin-1-ylacetyl)amino]-4,5,6,7-tetrahydro-1-benzothiophene-3-carboxylate;
ethyl 4-(4-bromophenyl)-2-[(piperidin-1-ylacetyl)amino]thiophene-3-carboxylate;
ethyl 5-[(piperidin-1-ylacetyl)amino]-2,3′-bithiophene-4-carboxylate;
ethyl 5-[(piperidin-1-ylacetyl)amino]-3,3′-bithiophene-4-carboxylate;
Isopropyl 2-[(piperidin-1-ylacetyl)amino]-4,5,6,7-tetrahydro-1-benzothiophene-3-carboxylate;
isopropyl 4-(3,4-dichlorophenyl)-2-[(piperidin-1-ylacetyl)amino]thiophene-3-carboxylate;
ethyl 2-[(piperidin-1-ylacetyl)amino]-4,5-dihydrothieno[2,3-b]thiophene-3-carboxylate;
ethyl 2-{[(4-hydroxypiperidin-1-yl)acetyl]amino}-4,5,6,7-tetrahydro-1-benzothiophene-3-carboxylate; and
ethyl 2-{[(3-hydroxypiperidin-1-yl)acetyl]amino}-4,5,6,7-tetrahydro-1-benzothiophene-3-carboxylate.

5. A composition comprising a compound of claim 1 and a pharmaceutically acceptable carrier.

6. A composition comprising a compound of claim 3 and a pharmaceutically acceptable carrier.

7. The use of a compound of claim 1 to prepare a medicament for treating or preventing parasitic diseases in mammals.

8. The use of a compound of claim 3 to prepare a medicament for treating or preventing parasitic diseases in mammals.
